(19) United States
(12) Patent Application Publication
(10) Pub. No.: US 2018/0001310 A1
(43) Pub. Date: Jan. 4, 2018

(54) Catalysts for Producing Isocyanurates from Isocyanates

(71) Applicant: Covestro Deutschland AG, Leverkusen (DE)

(72) Inventors: Thomas Ernst Müller, Aachen (DE); Torsten Hagen, Essen (DE); Bolko Raffel, Dormagen (DE); Christoph Gürtler, Köln (DE); Henning Vogt, Mülheim (DE); Abdulghani Al Nabulsi, Aachen (DE); Burkhard Köhler, Zierenberg (DE); Walter Leitner, Aachen (DE)

(21) Appl. No.: 15/534,790

(22) PCT Filed: Dec. 10, 2015
(86) PCT No.: PCT/EP2015/079254

§ 371 Date: Jun. 9, 2017

(30) Foreign Application Priority Data
Dec. 11, 2014 (EP) 14197329.7

(51) Int. Cl. B01J 31/02; C07D 251/30; C08G 18/10; C08G 18/16; C08G 18/40; C08J 9/14; C08G 18/76; C08G 18/08; C08G 101/00

(52) U.S. Cl. CPC B01J 31/0229; C08G 18/7671; C07D 251/30; C08G 18/10; C08G 18/14; C08G 18/168; C08G 18/4018; C08J 9/141; C08G 2101/0025; C08G 2330/00; B01J 2231/14; C08J 2203/14; C08J 2205/10; C08J 2375/06

(57) ABSTRACT

The invention relates to a method for producing isocyanurates and isocyanurate-containing polyurethanes, comprising the step of reacting an isocyanate in the presence of a catalyst.

The present invention describes a process for producing isocyanurates and isocyanurate-containing polyurethanes using isocyanates in the presence of a catalyst, wherein the catalyst comprises the product of the reaction of a thiol group containing carboxylic acid with an alkali metal and/or alkaline earth metal base.

Isocyanurates play an important role in the production of polyurethane foams. They may result from trimerization of the isocyanates used in the production of the polyurethane foam and provide the resulting foam with advantageous properties, for example high stiffness, high chemicals resistance and in particular advantageous fire behaviour.

Two reactions play an essential role in the production of isocyanurate-containing polyurethane foams from di- or polyisocyanates and di- or polyalcohols: the urethanization reaction of one of each of an isocyanate group and an alcohol group to afford urethane units (also interchangeably referred to as carbamate units hereinbelow) and the trimerization reaction of in each case three isocyanate groups to afford isocyanurate units (also interchangeably referred to as trimer units hereinbelow).

On account of the exothermicity of the chemical reaction of di- or polyisocyanates and di- or polyalcohols during adiabatic or polytropic foam formation, the foam formed undergoes heating to temperatures of up to 180° C. Onset of the urethanization reaction typically occurs even at moderate temperatures in an early phase of foam formation. Onset of the additionally desirable formation of isocyanurate units from the employed isocyanates typically only occurs at a higher temperature range, and thus with a time delay, when the urethanization reaction is already largely complete. As a result of the progressing polyurethane formation the viscosity of the foam continually increases during foam formation so that due to the increasing inflexibility of the surrounding medium the coming together of the remaining isocyanate groups to form isocyanurates is impeded.

One particular problem may arise in the edge regions of the foam during polyurethane foam formation. These typically have a markedly lower temperature than the foam core since the foam edges are in contact with the cooler environment or with colder parts of production plants. At the temperatures prevailing there during foam formation which are relatively low compared to the foam core a smaller proportion of isocyanurate units is formed. Accordingly, an inhomogeneous foam is obtained.

Special catalysts, such as potassium acetate or potassium 2-ethylhexanoate for example, are often employed to deliberately form isocyanurate units in the polyurethane foam.

Methods of achieving an advantageous concentration of isocyanurate structures even at low temperatures and/or in the edge regions of the polyurethane foam are the subject of research. Thus, WO2010054311, WO2010054313, WO2010054315, WO2010054317 describe the use of various phosphorus-/nitrogen-containing catalysts which have an activation temperature ≦73° C. for the isocyanurate formation reaction and are said to increase the yield of isocyanurate structures in the edge region. However, the catalysts used here also show a marked fall in isocyanurate structures in the edge region of the polyurethane foam (e.g. WO2010054317 A2, diagram on last page).

The customarily used catalysts exhibit low activity at temperatures below 70° C. Consequently, industrial production of isocyanurate-containing polyurethane foams typically requires the highest processing temperatures possible to ensure a sufficiently high rate of formation of the isocyanurate-containing polyurethane foam and a sufficient isocyanurate content. In the case of rigid foam sandwich panels this requires a high temperature of the double conveyor line (line temperature) which is often in the region of 70° C. However, a high line temperature results in increased energy requirements for heating the production plant.

In the search for novel isocyanurate formation catalysts, sulfur-containing compounds have been considered in connection with PUR/PIR systems only in certain aspects. In many cases sulfur-containing compounds are described as additives or reaction components. For example, Applied Polymer Science (2014) 131(13), 40402/1-40401/11 or Progress in Organic Coatings (2009) 64(2-3), 238-246 describe thiols for controlling elasticity in crosslinked urethane acrylates or pentaerythritol tetrakis(3-mercaptopropionate) and comparable compounds as reactants for the thiol-ene addition.

DE 2422647 A1 describes elemental sulfur as a flame retardant additive in isocyanurate-urethane foam mixtures.

The prior art further discloses sulfur-containing ligands for tin and lead catalysts: Dibutyltin bis(ethoxybutyl-3-mercaptopropionate) (WO 2008/089163 A1), dimethyltin bis(3-mercaptopropionate) (EP 0651017 A1), dibutyltin mercaptopropionate (EP 0075130 A1), dimethyltin dilauryl mercaptide (WO 2009/143198 A1), tin sulfides and tin thiolates (U.S. Pat. No. 6,613,865) and also triphenyllead thioacetate (U.S. Pat. No. 3,474,075). U.S. Pat. No. 4,173,692 A describes mixtures of carboxylates, which may also comprise mercapto groups, with tin catalysts but the mixtures are also heavy-metal-containing and the reaction rate and selectivity for trimer formation are not sufficient for some applications. WO 2013/117541 A1 describes a mixture of carboxylates, heavy metal compounds and special amines as a catalyst system. Improved flame retardancy is purported but foaming proceeds from a prepolymer, thus necessitating an upstream reaction step.

DD 121 461 A3 describes a heavy-metal-free sulfur-containing catalysis in the PUR/PIR field. This document relates to a process for producing polyisocyanurate-polycarbodiimide molding materials including polyisocyanurate-polycarbodiimide foams having increased heat resistance and flame retardancy and whose carbodiimide-containing isocyanate prepolymers are storage stable as intermediates, by reaction of polyisocyanates with organic oxygen-containing sulfur compounds which bring about partial formation of carbodiimide groups and subsequent trimerization. The carbodiimide-effecting catalyst used is dialkyl sulfide, dialkyl sulfate or dialkyl sulfite. EP 0,476,337 A1 describes a heavy-metal-free catalyst system composed of carboxylates and trisdialkylaminoalkylhexahydrotriazines. However this system is sulfur-free which has a negative effect on flame retardancy.

There remains a need for improved production processes for PUR/PIR foams that are less energy intensive. A particular concern is the temperature at which the reaction to afford the PUR/PIR foam is performed. It is furthermore desirable to improve the fire behaviour of PUR/PIR foams. The catalyst/the catalyst system should also be free from heavy metals, such as tin or lead.

The present invention has for its object the provision of a process for producing isocyanurates which can be performed at lower temperatures than has hitherto been customary and provides foams having improved fire behavior.

The object is achieved in accordance with the invention by a process for producing isocyanurates and isocyanurate-containing polyurethanes comprising the step of reacting an isocyanate in the presence of a catalyst, wherein the catalyst is the product of the reaction of a thiol group containing carboxylic acid with an alkali metal, alkaline earth metal, scandium-group or lanthanoid base, wherein the reaction is performed in the absence of compounds comprising tin and/or lead, wherein the degree of deprotonation of the catalyst is >50% to ≦100% and the H atoms present in carboxyl groups as well as the carboxylate groups and the H atoms present in thiol groups as well as the thiolate groups are considered when calculating the degree of deprotonation.

The process according to the invention has the advantage that isocyanurates/isocyanurate units and in particular mixtures of isocyanurates/isocyanurate units and carbamates/urethane units, as are present in isocyanurate-containing polyurethanes (PUR/PIR systems), are obtained at lower temperatures with higher reaction rates than in comparable processes in which catalysts not comprising mercapto groups are employed, for example potassium acetate.

The catalysts selected in accordance with the invention have the further advantage that at low temperatures, for example in a range around 40° C., increased activity in the conversion of isocyanate groups is observed compared to potassium acetate. It is a result of this increased activity that relatively large amounts of isocyanurates/isocyanurate units are formed even in an early stage of the reaction of isocyanates and alcohols/of di- or polyisocyanates and di- or polyalcohols. In this way polyurethane foams having an increased isocyanurate content, in particular in the edge regions of the foam, may moreover be obtained even at typically used line operating temperatures, such as 70° C. for example.

In addition, a higher relative reactivity (formation of trimer/formation of carbamate) is observed for example at 70° C. compared to potassium acetate.

As a result of the high activity of the catalysts according to the invention for formation of carbamate/isocyanurate mixtures, i.e. PUR/PIR systems, at low temperatures (for example in the range from 40° C. to 70° C.) the process according to the invention allows production of isocyanurate-containing polyurethane foams at low line operating temperatures (in the case of slabstock foam or panels), for example in the range from 40° C. to 70° C. This means that compared to conventional processes isocyanurate-containing polyurethane foams can be produced while saving energy required for heating the production line.

Independently of temperature a higher activity of the catalysts according to the invention for formation of carbamate/isocyanurate mixtures, i.e. PUR/PIR systems, has the result that polyurethane foams having a suitable isocyanurate content can be produced at lower reaction times, thus ensuring a higher productivity of the production plant.

An increased relative activity of the catalysts according to the invention for formation of isocyanurate units (versus formation of carbamate/urethane units), in particular at low temperatures (for example in the range of 40° C. to 70° C.) moreover makes it possible to produce PUR/PIR systems having an increased isocyanurate proportion and thus improved fire behavior.

The catalyst is regarded as the reaction product of a thiol group containing carboxylic acid with an alkali metal, alkaline earth metal, scandium-group or lanthanoid base.

Depending on the charge of the alkali metal, alkaline earth metal, scandium-group or lanthanoid cations (base cations) derived from the alkali metal, alkaline earth metal, scandium-group or lanthanoid base, the valency of the alkali metal, alkaline earth metal, scandium-group or lanthanoid base or more generally the total number of the positive charges present, the strength of the alkali metal, alkaline earth metal, scandium-group or lanthanoid base and the number of protons that are bound in COOH and SH groups and thus cleavable, a dianion of the thiol group containing carboxylic acid with two base monocations or a dianion of the thiol group containing carboxylic acid with one base dication may be present in the catalyst, for example.

It is likewise possible for mixtures of the aforementioned combinations to be present. It is further possible for the thiol group containing carboxylic acid to be present in a protonation/deprotonation equilibrium with the alkali metal or alkaline earth metal base.

The thiol group containing carboxylic acid may for example be an aliphatic or aromatic carboxylic acid bonded to at least one thiol group via the aliphatic or aromatic radical. A plurality of carboxyl groups and/or a plurality of thiol groups may be present in the molecule.

The alkali metal or alkaline earth metal base may consist of the combination of a base anion Bⁿ⁻ with a suitable number of alkali metal or alkaline earth metal cations M^{m+} and typically has the composition (M^{m+})_{b}(B^{b-})ₘ, wherein m represents 1 or 2 and b represents 1, 2 or 3 and corresponds to the valency of the base. In the alkali metal or alkaline earth metal base having the above composition the cations M^{m+} may be partially replaced by protons H⁺, wherein the total charge of the cations to be replaced corresponds to the total charge of the protons replacing them and at least one cation M^{m+} is present in the alkali metal or alkaline earth metal base.

The cations M^{m+} may be selected from the group of the alkali metals, in particular lithium, sodium, potassium, rubidium, cesium, from the group of the alkaline earth metals, in particular magnesium, calcium, strontium, barium, from the scandium group, in particular scandium, yttrium or from the group of the lanthanoids, in particular lanthanum, europium, gadolinium, ytterbium, lutetium.

The base anions B^{b-} may for example be monovalent base anions such as H⁻, OH⁻, OOH⁻, SH⁻, ClO⁻, CN⁻, alkoxides, thiolates, amides, carboxylates, carbanions for example or divalent base anions such as O²⁻, CO₃²⁻, SO₃²⁻, HPO₄²⁻ for example or trivalent base anions such PO₄³⁻ for example.

The alkali metal, alkaline earth metal, scandium-group or lanthanoid base may furthermore be an elemental alkali metal, alkaline earth metal, scandium group metal or lanthanoid metal.

It is provided according to the invention that the reaction is performed in the absence of compounds comprising tin or lead. Compounds to be avoided are in particular dibutyltin dilaurate (DBTL), dibutyltin bis(ethoxybutyl-3-mercaptopropionate), dimethyltin bis(3-mercaptopropionate), dibutyltin mercaptopropionate, dimethyltin dilauryl mercaptide, tin sulfides and tin thiolates and also triphenyllead thioacetate. Compounds comprising bismuth, for example bismuth trioctoate, are preferably likewise excluded.

It is further provided in accordance with the invention that the degree of deprotonation of the catalyst is >50% to ≦100%, wherein the H atoms present in carboxyl groups as well as the carboxylate groups and the H atoms present in thiol groups as well as the thiolate groups are considered when calculating the degree of deprotonation.

The degree of deprotonation may be derived from the employed amounts of thiol group containing carboxylic acids and alkali metal, alkaline earth metal, scandium group or lanthanoid bases. The proton bonded to the COOH group is generally more acidic than the proton bonded to the thiol group and will react with the base first. Only afterwards will the proton bonded to the SH group react.

The degree of deprotonation is to be understood as meaning the percentage of Zerewittinoff-active protons removed from the acid upon which the catalyst molecule is based. Zerewittinoff-active protons are those that react with the Grignard reagent methylmagnesium iodide to form one molecule of methane per active proton.

Under this premise a degree of deprotonation of 50% indicates that, when employing a thiol group containing carboxylic acid in which the number of carboxyl groups present is equal to the number of thiol groups present, all carboxyl groups are present in deprotonated form and all thiol groups are present in protonated form.

A degree of deprotonation of 100% accordingly indicates that all carboxyl and thiol groups present in the thiol group containing carboxylic acid are present in deprotonated form.

The degree of deprotonation may be determined by analysis of the ratio of alkali metal, alkaline earth metal, scandium-group or lanthanoid cations to sulfur by elemental analysis The degree of deprotonation is then given by equation 1 which follows,

$$Degree of deprotonation = m \times \frac{M}{S} \times \frac{1}{1 + \frac{{n}_{COOH}}{{n}_{SH}}} \times 100 \% \qquad ( Eq . 1 )$$

wherein m represents the charge of the alkali metal, alkaline earth metal, scandium-group or lanthanoid cation, M/S represents the molar ratio of alkali metal, alkaline earth metal, scandium-group metal or lanthanoid metal M to sulfur, as determined by elemental analysis, and n_{COOH}/n_{SH} represents the ratio of carboxyl groups to thiol groups in the thiol group containing carboxylic acid.

Embodiments and further aspect of the invention are described hereinbelow. They may be combined with one another as desired unless the opposite is unequivocally clear from the context.

The process according to the invention may moreover employ further catalysts, for example urethanization catalysts. Examples of such urethanization catalysts are aminic catalysts, in particular selected from the group of triethylenediamine, N,N-dimethylcyclohexylamine, dicyclohexylmethylamine, tetramethylenediamine, 1-methyl-4-dimethylaminoethylpiperazine, triethylamine, tributylamine, N,N-dimethylbenzylamine, N,N′,N″-tris(dimethylaminopropyl)hexahydrotriazine, tris(dimethylaminopropyl)amine, tris(dimethylaminomethyl)phenol, dimethylaminopropylformamide, N,N,N′,N′-tetramethylethylenediamine, N,N,N′,N′-tetramethylbutanediamine, tetramethylhexanediamine, pentamethyldiethylenetriamine, pentamethyldipropylenetriamine, tetramethyldiaminoethyl ether, dimethylpiperazine, 1,2-dimethylimidazole, 1-azabicyclo[3.3.0]octane, 1,4-diazabicyclo[2.2.2]octane, bis(dimethylaminopropyl)urea, N-methylmorpholine, N-ethylmorpholine, N-cyclohexylmorpholine, 2,3-dimethyl-3,4,5,6-tetrahydropyrimidine, triethanolamine, diethanolamine, triisopropanolamine, N-methyldiethanolamine, N-ethyldiethanolamine and/or dimethylethanolamine

In a further embodiment of the present invention the thiol group containing carboxylic acid comprises a thiol group and a carboxyl group.

In a further embodiment of the present invention the thiol group containing carboxylic acid comprises one thiol group and one carboxyl group and the thiol group and the carboxyl group are bridged via not more than 3 carbon atoms, wherein “bridging carbon atoms” is to be understood as meaning the chain having the fewest carbon atoms between the carboxyl group and the thiol group in the molecule and the carbon atom present in the carboxyl group is not considered. Examples of thiol group containing carboxylic acids of this embodiment are 2-mercaptoacetic acid, 3-mercaptopropionic acid, 4-mercaptobutyric acid, wherein the carbon atoms in the bridging alkylene chain may each independently of one another be bonded to further radicals, for example linear or branched, saturated or mono- or polyunsaturated, optionally heteroatom-containing C1- to C20-alkyl, cycloalkyl, aryl, alkylaryl or arylalkyl groups, fluorine, chlorine or bromine atoms, nitrile groups and/or nitro groups and different radicals may be bonded to one another such that they form mono-, bi-oder polycyclic ring systems, and thiosalicylic acid, wherein the aromatic carbon atoms not bonded to the thiol group or to the carboxyl group may each independently of one another be bonded to further radicals, for example linear or branched, saturated or mono- or polyunsaturated, optionally heteroatom-containing C1- to C20-alkyl, cycloalkyl, aryl, alkylaryl or arylalkyl groups, fluorine, chlorine or bromine atoms, nitrile groups and/or nitro groups and/or may be fused together to form higher ring systems. Preferred thiol group containing carboxylic acids in this embodiment are 2-mercaptoacetic acid, 2-mercaptopropionic acid, 4-mercaptobutyric acid and thiosalicylic acid.

In a further embodiment of the present invention the thiol group containing carboxylic acid comprises one thiol group and one carboxyl group and the thiol group and the carboxyl group are bridged via 2 or 3 carbon atoms, wherein “bridging carbon atoms” is to be understood as meaning the chain having the fewest carbon atoms between the carboxyl group and the thiol group in the molecule and the carbon atom present in the carboxyl group is not considered. Examples of the thiol group containing carboxylic acids of this embodiment are 3-mercaptopropionic acid, 4-mercaptobutyric acid, wherein the carbon atoms in the bridging alkylene chain may each independently of one another be bonded to further radicals, for example linear or branched, saturated or mono- or polyunsaturated, optionally heteroatom-containing C1- to C20-alkyl, cycloalkyl, aryl, alkylaryl or arylalkyl groups, fluorine, chlorine or bromine atoms, nitrile groups and/or nitro groups and different radicals may be bonded to one another such that they form mono-, bi-oder polycyclic ring systems, and thiosalicylic acid, wherein the aromatic carbon atoms not bonded to the thiol group or to the carboxyl group may each independently of one another be bonded to further radicals, for example linear or branched, saturated or mono- or polyunsaturated, optionally heteroatom-containing C1- to C20-alkyl, cycloalkyl, aryl, alkylaryl or arylalkyl groups, fluorine, chlorine or bromine atoms, nitrile groups and/or nitro groups and/or may be fused together to form higher ring systems. Preferred thiol group containing carboxylic acids in this embodiment are 2-mercaptopropionic acid, 4-mercaptobutyric acid and thiosalicylic acid.

In a further embodiment of the present invention the thiol group containing carboxylic acid is 2-mercaptoacetic acid, 3-mercaptopropionic acid, 4-mercaptobutyric acid and/or thiosalicylic acid. Preferred catalysts are the dipotassium salts of the recited acids.

In a further embodiment of the present invention the degree of deprotonation of the catalyst is ≧70% to ≦100%, wherein the H atoms present in carboxyl groups as well as the carboxylate groups and the H atoms present in thiol groups as well as the thiolate groups are considered when calculating the degree of deprotonation. The degree of deprotonation is calculated as previously described hereinabove. The degree of deprotonation is preferably ≧80% to ≦100%, more preferably ≧90% to ≦100% and particularly preferably ≧95% to ≦100%.

In a further embodiment the base for deprotonating the catalyst precursor is an alkali metal, alkaline earth metal, scandium-group or lanthanoid hydride, an alkali metal, alkaline earth metal, scandium-group or lanthanoid alkoxide or an or an alkali metal, alkaline earth metal, scandium-group or lanthanoid alkyl.

The advantage of using hydrides as the base is that gaseous hydrogen escapes as a byproduct and thus no neutralization products such as water are present in the reaction mixture. Preference is given to lithium hydride, sodium hydride, potassium hydride, magnesium hydride and/or calcium hydride.

The alkali metal, alkaline earth metal, scandium-group or lanthanoid alkoxides may be obtained for example by reaction of a suitable alkali metal/alkaline earth metal base, in particular of an alkali metal, alkaline earth metal, scandium-group or lanthanoid hydride or of an elemental alkali metal, alkaline earth metal, scandium-group metal or lanthanoid metal with the corresponding alcohol. Examples of alcohols which may be reacted with alkali metal, alkaline earth metal, scandium-group or lanthanoid bases to afford suitable alkoxides are methanol, ethanol, n-propanol, isopropanol, n-butanol, isobutanol, tert-butanol, n-pentanol, tert-pentanol, neopentyl alcohol, cyclopentanol, hexanol, cyclohexanol, heptanol, octanol, 2-ethylhexanol, nonanol, decanol, undecanol, dodecanol and the higher homologues thereof, monomeric, oligomeric or polymeric diols, in particular alkylene, oligoalkylene or polyalkylene glycols, for example ethylene glycol, diethylene glycol, triethylene glycol, polyethylene glycol, dipropylene glycol, tripropylene glycol, polypropylene glycol, tetramethylene glycol, di(tetramethylene glycol), poly(tetramethylene glycol), and monoalkyl ethers, in particular monomethyl, monoethyl, monopropyl and monobutyl ethers of monomeric, oligomeric or polymeric diols. Preferred alcohols therefor are methanol, ethanol, n-propanol, isopropanol, n-butanol, isobutanol, tert-butanol, n-pentanol, tert-pentanol, neopentyl alcohol, diethylene glycol, diethylene glycol monomethyl ether or a polyalkylene glycol/the monomethyl ether thereof. The alkali metal, alkaline earth metal, scandium-group or lanthanoid alkoxide may be present in the form of a solution in a solvent, for example in one of the abovementioned alcohols.

The advantage of using carbanions as the base is that chemically inert compounds are formed as a byproduct and thus no neutralization products such as water are present in the reaction mixture. Preference is given to methyllithium, ethyllithium, methylsodium, ethylsodium, methylpotassium, ethylpotassium and/or methylmagnesium chloride.

In a further embodiment of the present invention the catalyst is present in the form of a solution or a suspension in a solvent before commencement of the reaction. The solvent is preferably monoethylene glycol, diethylene glycol, diethylene glycol monomethyl ether or a polyalkylene glycol, N-methylpyrrolidone, N-ethylpyrrolidone or dimethylsulfoxide or mixtures thereof. The catalyst preparations may additionally comprise further constituents, for example monofunctional alcohols. Without wishing to be bound to a particular theory it is believed that the recited compounds exert an influence on the catalytic system according to the invention at least as labile ligands.

In a further embodiment of the present invention, the reaction is performed at a temperature of ≧20° C. to ≦90° C. Preferred reaction temperatures are ≧30° C. to ≦80° C., particularly preferably ≧40° C. to ≦70° C.

In a further embodiment of the present invention the reaction is performed in a non-constant temperature range, a temperature of ≧20° C. to ≦90° C., preferably ≧30° C. to ≦80° C., particularly preferably ≧40° C. to ≦70° C., prevailing at commencement of the reaction however. In this embodiment after onset of the reaction a temperature rise in the reaction system is typically observed so that the maximum temperature of the reaction system may be ≧80° C. to ≦250° C., preferably ≧100° C. to ≦220° C., particularly preferably ≧140° C. to ≦200° C. Such an adiabatic temperature profile is typically observed in particular in PUR/PIR systems, i.e. for the reaction of diisocyanates and/or polyisocyanates with diols and/or polyols. In the case of slabstock foam or panels the temperature at commencement of the reaction is the line operating temperature. Input materials, for example isocyanates, alcohols, catalyst solution and other components of the foam formulation may be preheated to this temperature or a lower temperature prior to mixing at commencement of the reaction.

In one embodiment of the present invention the isocyanate is a monoisocyanate. When monoisocyanates are used monomeric isocyanurates are obtained. Examples of monoisocyanates are methyl isocyanate, ethyl isocyanate, n-propyl isocyanate, isopropyl isocyanate, n-butyl isocyanate, isobutyl isocyanate, tent-butyl isocyanate, n-pentyl isocyanate, n-hexyl isocyanate, cyclohexyl isocyanate, ω-chlorohexamethylene isocyanate, n-heptyl isocyanate, n-octyl isocyanate, isooctyl isocyanate, 2-ethylhexyl isocyanate, 2-norbornylmethyl isocyanate, nonyl isocyanate, 2,3,4-trimethylcyclohexyl isocyanate, 3,3,5-trimethylcyclohexyl isocyanate, decyl isocyanate, undecyl isocyanate, dodecyl isocyanate, tridecyl isocyanate, tetradecyl isocyanate, pentadecyl isocyanate, hexadecyl isocyanate, octadecyl isocyanate, stearyl isocyanate, 3-butoxypropyl isocyanate, 3-(2-ethylhexyloxy)propyl isocyanate, 6-chlorohexyl isocyanate, benzyl isocyanate, phenyl isocyanate, ortho-, meta-, para-tolyl isocyanate, dimethylphenyl isocyanate (technical mixtures and individual isomers), 4-pentylphenyl isocyanate, 4-cyclohexylphenyl isocyanate, 4-dodecylphenyl isocyanate, ortho-, meta-, para-methoxyphenyl isocyanate, chlorophenyl isocyanate (2,3,4-isomer), the various dichlorophenyl isocyanate isomers, 4-nitrophenyl isocyanate, 3-trifluormethylphenyl isocyanate, 1-naphthyl isocyanate. Preferred monoisocyanates are benzyl isocyanate, phenyl isocyanate, ortho-, meta-, paratolyl isocyanate, dimethylphenyl isocyanate (technical mixtures and individual isomers), 4-cyclohexylphenyl isocyanate and ortho-, meta-, para-methoxyphenyl isocyanate. A particularly preferred monoisocyanate is para-tolyl isocyanate.

In a further embodiment of the present invention the isocyanate is a polyisocyanate. These are isocyanates customary in the PUR/PIR field having an NCO functionality of 2 or more. Generally contemplated are aliphatic, cycloaliphatic, arylaliphatic and aromatic polyfunctional polyisocyanates. Examples of suitable polyisocyanates of this type include 1,4-butylene diisocyanate, 1,5-pentane diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof with any desired isomer content, 1,4-cyclohexylene diisocyanate, 1,4-phenylene diisocyanate, 2,4- and/or 2,6-tolylene diisocyanate (TDI), 1,5-naphthylene diisocyanate, 2,2′- and/or 2,4′- and/or 4,4′-diphenylmethane diisocyanate (MDI) or a higher homologous or mixtures thereof (polymeric MDI), 1,3- and/or 1,4-bis(2-isocyanatoprop-2-yl)benzene (TMXDI), 1,3-bis(isocyanatomethyl)benzene (XDI), and also alkyl 2,6-diisocyanatohexanoates (lysine diisocyanates) having C₁ to C₆-alkyl groups. An isocyanate from the diphenylmethane diisocyanate series is preferred.

Employable polyisocyanates further include NCO-terminated prepolymers obtainable for example from the reaction of one of the above mentioned polyisocyanates with polyols, in particular polyalkylene glycols.

In addition to the abovementioned polyisocyanates, it is also possible to employ proportions of modified diisocyanates of uretdione, isocyanurate, urethane, carbodiimide, uretoneimine, allophanate, biuret, iminooxadiazinedione and/or oxadiazinetrione structure and also unmodified polyisocyanate having more than 2 NCO groups per molecule, for example 4-isocyanatomethyl-1,8-octane diisocyanate (nonane triisocyanate), tris-4-isocyanatophenyl thiophosphate or triphenylmethane 4,4′,4″-triisocyanate.

In a further embodiment the reaction is further performed in the presence of a monoalcohol. The use of monoisocyanates thus makes it possible to obtain mixtures of monomeric isocyanurates with carbamates which result from the reaction of the monoisocyanates with the alcohol. The composition of the mixture depends on the nature of the monoisocyanate and of the monoalcohol, on the ratio of isocyanate groups to hydroxyl groups present in the alcohol, on the nature and concentration of the catalyst and on the reaction conditions such as temperature, solvent and reaction management. Examples of monoalcohols are methanol, ethanol, n-propanol, isopropanol, n-butanol, isobutanol, tent-butanol, n-pentanol, tert-pentanol, neopentyl alcohol, cyclopentanol, hexanol, cyclohexanol, heptanol, octanol, 2-ethylhexanol, nonanol, decanol, undecanol, dodecanol and the higher homologues thereof, monoalkyl ether of monomeric, oligomeric or polymeric diols, for example ethylene glycol monomethyl ether, ethylene glycol monoethyl ether, ethylene glycol monopropyl ether, ethylene glycol monobutyl ether, diethylene glycol monomethyl ether, diethylene glycol monoethyl ether, diethylene glycol monopropyl ether, diethylene glycol monobutyl ether, triethylene glycol monomethyl ether, triethylene glycol monoethyl ether, triethylene glycol monopropyl ether, triethylene glycol monobutyl ether, polyethylene glycol monomethyl ether, polyethylene glycol monoethyl ether, polyethylene glycol monopropyl ether, polyethylene glycol monobutyl ether, propylene glycol monomethyl ether, propylene glykol monoethyl ether, propylene glycol monopropyl ether, propylene glycol monobutyl ether, dipropylene glycol monomethyl ether, dipropylene glycol monoethyl ether, dipropylene glycol monopropyl ether, dipropylene glycol monobutyl ether, tripropylene glycol monomethyl ether, tripropylene glycol monoethyl ether, tripropylene glycol monopropyl ether, tripropylene glycol monobutyl ether, polypropylene glycol monomethyl ether, polypropylene glycol monoethyl ether, polypropylene glycol monopropyl ether, polypropylene glycol monobutyl ether, tetramethylene glycol monomethyl ether, tetramethylene glycol monoethyl ether, tetramethylene glycol monopropyl ether, tetramethylene glycol monobutyl ether, di(tetramethylene glycol) monomethyl ether, di(tetramethylene glycol) monoethyl ether, di(tetramethylene glycol) monopropyl ether, di(tetramethylene glycol) monobutyl ether, poly(tetramethylene glycol) monomethyl ether, poly(tetramethylene glycol) monoethyl ether, poly(tetramethylene glycol) monopropyl ether, poly(tetramethylene glycol) monobutyl ether.

Preferred monoalcohols are primary alcohols, for example methanol, ethanol, n-propanol, n-butanol, n-pentanol, tert-pentanol, neopentyl alcohol, n-hexanol, n-octanol, 2-ethylhexanol, ethylene glycol monomethyl ether, ethylene glycol monoethyl ether, ethylene glycol monopropyl ether, ethylene glycol monobutyl ether, diethylene glycol monomethyl ether, diethylene glycol monoethyl ether, diethylene glycol monopropyl ether, diethylene glycol monobutylether, triethylene glycol monomethyl ether, triethylene glycol monoethyl ether, triethylene glycol monopropyl ether, triethylene glycol monobutyl ether, polyethylene glycol monomethyl ether, polyethylene glycol monoethyl ether, polyethylene glycol monopropyl ether, polyethylene glycol monobutyl ether. Particularly preferred monoalcohols are monoalkyl ethers of diols, in particular ethylene glycol monomethyl ether, ethylene glycol monoethyl ether, ethylene glycol monopropyl ether, ethylene glycol monobutyl ether, diethylene glycol monomethyl ether, diethylene glycol monoethyl ether, diethylene glycol monopropyl ether, diethylene glycol monobutyl ether, triethylene glycol monomethyl ether, triethylene glycol monoethyl ether, triethylene glycol monopropyl ether, triethylene glycol monobutyl ether, polyethylene glycol monomethyl ether, polyethylene glycol monoethyl ether, polyethylene glycol monopropyl ether, polyethylene glycol monobutyl ether.

In a preferred embodiment the reaction is further performed in the presence of a polyol. The polyols employable in accordance with the invention may have, for example, a number-average molecular weight Mₙ of ≧60 g/mol to ≦8000 g/mol, preferably of ≧90 g/mol to ≦5000 g/mol and more preferably of ≧92 g/mol to ≦1000 g/mol. In the case of a single added polyol the OH number of the polyols indicates the OH number of said polyol. In the case of mixtures the average OH number is reported. This value may be determined in accordance with DIN 53240. The average OH functionality of the recited polyols is ≧1.5 and is for example in a range of ≧1.5 to ≦6, preferably of ≧1.6 to ≦5 and more preferably of ≧1.8 to ≦4.

Polyether polyols usable in accordance with the invention are, for example, polytetramethylene glycol polyethers, as obtainable by polymerization of tetrahydrofuran by means of cationic ring opening.

Likewise suitable polyether polyols are addition products of styrene oxide, ethylene oxide, propylene oxide, butylene oxides and/or epichlorohydrin onto di- or polyfunctional starter molecules.

Suitable starter molecules are for example water, ethylene glycol, diethylene glycol, butyl diglycol, glycerol, diethylene glycol, trimethylolpropane, propylene glycol, pentaerythritol, sorbitol, sucrose, ethylenediamine, toluenediamine, triethanolamine, 1,4-butanediol, 1,6-hexanediol and low molecular weight hydroxyl-containing esters of such polyols with dicarboxylic acids.

Polyester polyols usable in accordance with the invention are inter alia polycondensates of di- and also tri- and tetraols and di- and also tri- and tetracarboxylic acids or hydroxycarboxylic acids or lactones. Instead of the free polycarboxylic acids, it is also possible to use the corresponding polycarboxylic anhydrides or corresponding polycarboxylic esters of lower alcohols to produce the polyesters.

Examples of suitable diols are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate. In addition, it is also possible to use polyols such as trimethylolpropane, glycerol, erythritol, pentaerythritol, trimethylolbenzene or trishydroxyethyl isocyanurate.

Examples of polycarboxylic acids that may be used include phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, succinic acid, 2-methylsuccinic acid, 3,3-diethylglutaric acid, 2,2-dimethylsuccinic acid, dodecanedioic acid, endomethylenetetrahydrophthalic acid, dimer fatty acid, trimer fatty acid, citric acid, or trimellitic acid. Acid sources that may be used further include the corresponding anhydrides.

Co-use of aromatic monocarboxylic acids, for example benzoic acid, and/or aliphatic saturated or unsaturated monocarboxylic acids, for example fatty acids/mixtures thereof, is also possible.

Hydroxycarboxylic acids that may be co-used as reaction participants in the production of a polyester polyol having terminal hydroxyl groups are for example hydroxycaproic acid, hydroxybutyric acid, hydroxydecanoic acid, hydroxystearic acid and the like. Suitable lactones are inter alia caprolactone, butyrolactone and homologs.

Polycarbonate polyols usable in accordance with the invention are hydroxyl-containing polycarbonates, for example polycarbonate diols. These are obtainable by reaction of carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, dipropylene glycol, polypropylene glycols, dibutylene glycol, polybutylene glycols, bisphenol A and lactone-modified diols of the abovementioned type.

Instead of or in addition to pure polycarbonate diols, polyether-polycarbonate diols may also be used.

Polyetherester polyols usable in accordance with the invention are compounds containing ether groups, ester groups and OH groups. Organic dicarboxylic acids having up to 12 carbon atoms are suitable for producing the polyetherester polyols, preferably aliphatic dicarboxylic acids having ≧4 to ≦6 carbon atoms or aromatic dicarboxylic acids used singly or in admixture. Examples include suberic acid, azelaic acid, decanedicarboxylic acid, maleic acid, malonic acid, phthalic acid, pimelic acid and sebacic acid and in particular glutaric acid, fumaric acid, succinic acid, adipic acid, phthalic acid, terephthalic acid and isoterephthalic acid. Derivatives of these acids that may be used include, for example, their anhydrides and also their esters and monoesters with low molecular weight monofunctional alcohols having ≧1 to ≦4 carbon atoms.

Polyether polyols obtained by alkoxylation of starter molecules such as polyhydric alcohols are a further component used for producing polyetherester polyols. The starter molecules are at least difunctional, but may optionally also contain proportions of higher-functional, in particular trifunctional, starter molecules.

Starter molecules are for example diols having primary OH-groups and number-average molecular weights Mₙ of preferably ≧18 g/mol to ≦400 g/mol or of ≧62 g/mol to ≦200 g/mol such as 1,2-ethanediol, 1,3-propanediol, 1,4-butanediol, 1,5-pentenediol, 1,5-pentanediol, neopentylglycol, 1,6-hexanediol, 1,7-heptanediol, 1,8-octanediol, 1,10-decanediol, 2-methyl-1,3 -propanediol, 2,2-dimethyl-1,3 -propanediol, 3-methyl-1,5-pentanediol, 2-butyl-2-ethyl-1,3-propanediol, 2-butene-1,4-diol and 2-butyne-1,4-diol, ether diols such as diethylene glycol, triethylene glycol, tetraethylene glycol, dibutylene glycol, tributylene glycol, tetrabutylene glycol, dihexylene glycol, trihexylene glycol, tetrahexylene glycol and oligomeric mixtures of alkylene glycols, such as diethylene glycol.

In addition to the diols, polyols having number-average functionalities of >2 to ≦8, or of ≧3 to ≦4 may also be employed, examples being 1,1,1-trimethylolpropane, triethanolamine, glycerol, sorbitol, sorbitan and pentaerythritol and also triol- or tetraol-started polyethylene oxide polyols having average molecular weights of preferably ≧18 g/mol to ≦400 g/mol or of ≧62 g/mol to ≦200 g/mol.

Polyacrylate polyols usable in accordance with the invention are obtainable by free-radical polymerization of hydroxyl-containing, olefinically unsaturated monomers or by free-radical copolymerization of hydroxyl-containing, olefinically unsaturated monomers optionally with other olefinically unsaturated monomers. Examples thereof are ethyl acrylate, butyl acrylate, 2-ethylhexyl acrylate, isobornyl acrylate, methyl methacrylate, ethyl methacrylate, butyl methacrylate, cyclohexyl methacrylate, isobornyl methacrylate, styrene, acrylic acid, acrylonitrile and/or methacrylonitrile. Suitable hydroxyl-containing, olefinically unsaturated monomers are in particular 2-hydroxyethyl acrylate, 2-hydroxyethyl methacrylate, the hydroxypropyl acrylate isomer mixture obtainable by addition of propylene oxide onto acrylic acid, and the hydroxypropyl methacrylate isomer mixture obtainable by addition of propylene oxide onto methacrylic acid. Terminal hydroxyl groups may also be in protected form. Suitable free-radical initiators are those from the group of the azo compounds, for example azoisobutyronitrile (AIBN), or from the group of the peroxides, for example di-tert-butyl peroxide, dicumyl peroxide, dibenzoyl peroxide or tert.-butyl peroctoate.

In a further embodiment of the present invention the reaction is further performed in the presence of a physical blowing agent and/or a chemical blowing agent. This allows PUR/PIR foams to be obtained. Chemical blowing agents such as water, formic acid and also physical blowing agents such as hydrocarbon blowing agent (in particular n-pentane, i-pentane and cyclopentan and mixtures thereof) are conceivable.

In a further embodiment of the present invention the reaction is performed at an NCO index of ≧200. The NCO index is defined as 100 times the molar ratio of NCO groups in the polyisocyanate to isocyanate-reactive groups of the polyol component. This index may also be in a range of ≧250 to ≦500 or else of ≧300 to ≦400.

A further aspect of the present invention is a polyurethane/polyisocyanurate foam produced by a process according to the invention.

The polyurethane/polyisocyanurate foam preferably has a combustibility index CI of 5 and a flame height of ≦135 mm (more preferably ≦130 mm) in each case determined in the BVD test as per the Swiss Basic Test for Determination of Combustibility of Building Materials from the Vereinigung kantonaler Feuerversicherungen [Association of Cantonal Fire Insurers] in the edition of 1988, with the supplements of 1990, 1994, 1995 and 2005.

The invention finally further relates to a thermal insulation element comprising a polyurethane/polyisocyanurate foam according to the invention. What is preferably concerned here is a insulation panel laminated with covering layers, wherein the covering layers may be made for example of steel, aluminum, kraft paper or other materials. Processes for producing such thermal insulation elements are known and described for example in Günter Oertel, Polyurethane Handbook, Carl Hanser Verlag, Munchen, 1985, p 239f.

### EXAMPLES

The present invention is elucidated in detail by the figures and examples which follow, but without being limited thereto.

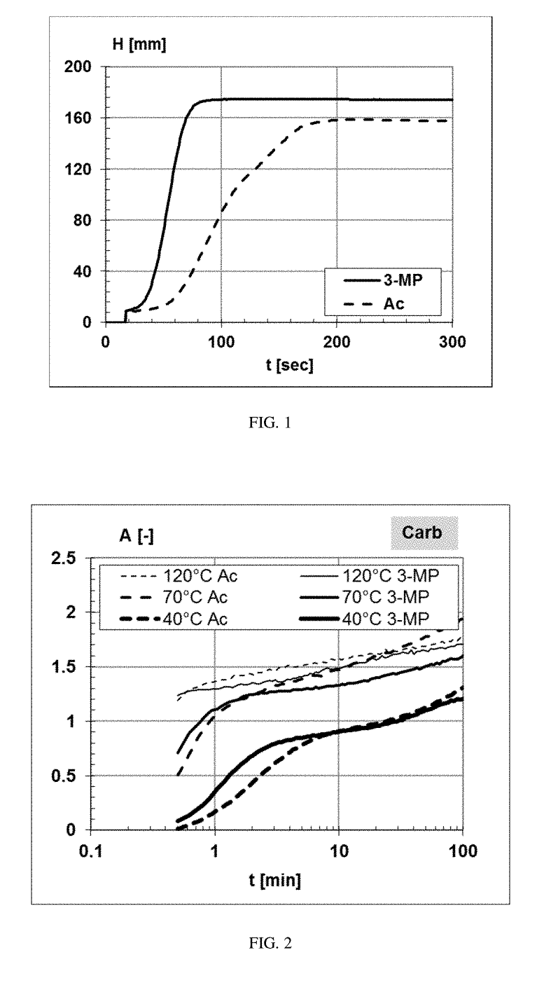

FIG. 1 shows a measurement of foam height from example 2-1* (Ac) and 2-2 (3-MP) as a function of time in the foaming apparatus from Format which is fitted with the “Advanced Test Cylinder” (ATC). The ATC and the instrument bottom had been temperature-controlled to 50° C.

FIG. 2 shows a time-resolved ATR mid-infrared spectrum of the Ac- and 3-MP-catalyzed foam systems from FIG. 1. The development of the carbamate-specific peak area with time (amide III between 1170 and 1250 cm⁻¹) is shown.

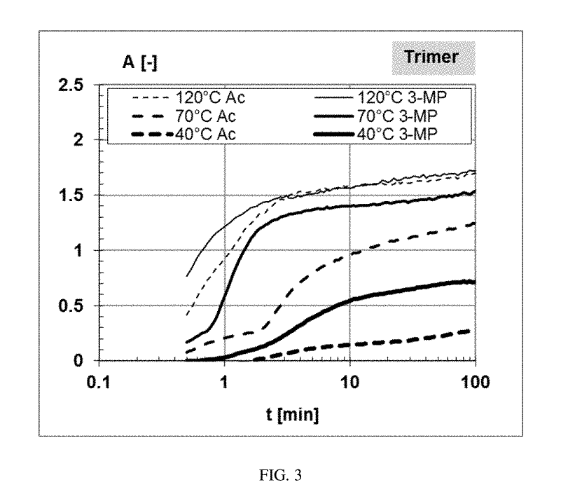

FIG. 3 shows a time-resolved ATR mid-infrared spectrum of the Ac- and 3-MP-catalyzed foam systems from FIG. 1. The development of the trimer-specific peak area with time (isocyanurate ring vibration between 1380 and 1450 cm⁻¹) is shown.

### METHODS

In-situ infrared spectroscopy: The composition of the reaction mixture as a function of time was monitored with a Bruker MATRIX-MX spectrometer. The infrared (IR) spectrometer was fitted with a high-pressure ATR (attenuated total reflectance) IR fiber optic probe (diameter 3.17 mm). The ATR-IR fiber optic probe (90° diamond prism with 1×2 mm basal area and 1 mm height as ATR element, 2×45° reflection of the IR beam, IR-beam-coupled fiber optics) was introduced into the reactor used in the reaction such that the diamond at the end of the probe was completely immersed in the reaction mixture. The IR spectra (20 scans per measurement) were acquired in a time-resolved manner at a scan rate of 266.7 scans per minute in the range of 400-4000 cm⁻¹ at a resolution of 4 cm⁻¹ at 4.5 second time intervals. An argon background spectrum (100 scans) was acquired at the beginning of each experiment. OPUS 7.0 software was used for recording the spectra.

Quantitative evaluation of the measured IR spectra was by means of PEAXACT 3.5.0 Software for Quantitative Spectroscopy from S•PACT GmbH using the Integrated Hard Model (IHM) method. The Hard Model for the product mixture was generated from the characteristic IR absorption bands of the individual components isocyanate, isocyanurate and carbamate. To achieve quantitative determination of the concentration of the individual components in the reaction mixture a calibration with known concentrations of the individual components at the respective reaction temperature was effected.

The time-resolved measurements in the reacting foam system (cf. FIGS. 2 and 3) were effected in a Bruker Tensor 27—spectrometer on a ZnSe ATR crystal of 1×5 cm² in size embedded in a heated metal plate at a constant controlled temperature of 40° C., 70° C. or 120° C. The reaction sequences in the approx. 1-μm-thick contact zone of the foam material with the ATR crystal at the established temperature are monitored therewith (spectral resolution 4 cm⁻¹; average over 8 scans).

The BVD test as per the Swiss Basic Test for Determination of Combustibility of Building Materials from the Vereinigung kantonaler Feuerversicherungen [Association of Cantonal Fire Insurers] in the edition of 1988, with the supplements of 1990, 1994, 1995 and 2005 (available from Vereinigung kantonaler Feuerversicherungen, Bundesstr. 20, 3011 Bern, Switzerland) was used as a basis for describing fire behavior. In this small burner test a combustibility index (CI) and a flame height (in mm) is determined for the foam.

Compounds Used:

Unless otherwise stated the catalysts employed were produced as follows from the corresponding catalyst precursor (thiol group containing carboxylic acid: 3-mercaptopropionic acid, 2-mercaptoacetic acid, 4-mercaptobutyric acid, o-thiosalicylic acid, S-methylthiosalicylic acid):

Production of the Catalyst as Solid

Under an argon atmosphere a solution of the catalyst precursor (0.01 mol) in anhydrous methanol (15 mL) was initially charged and at 25° C. a 25% solution of potassium methoxide in methanol (0.745 g, corresponding to 2.66 mmol, for forming the disalts and 0.372 g, corresponding to 1.33 mmol, for forming the monosalt) was added dropwise. The obtained reaction mixture was stirred at 25° C. for 30 minutes. This was followed by addition of anhydrous diithyl ether (10 mL) to precipitate-out a colorless solid. The supernatant solution was filtered off via a filter cannula and the solid filtration residue was washed three times with 10 mL respectively of a 1:5 mixture of anhydrous methanol and anhydrous diethyl ether. The thus obtained solid was dried for 16 h at 60° C. under vacuum (2.0×10⁻² mbar).

Production of the Catalyst Solution in Diethylene Glycol Monomethyl Ether (DEME)

An 11.2 percent solution of the solid-form catalysts in diethylene glycol monomethyl ether (DEME) was produced.

### Examples 1-1 to 1-10

### Production of Isocyanurates from p-tolyl Isocyanate in the Presence of Diethylene Glycol Monomethyl Ether

All reactions explicated in examples 1-1 to 1-10 were performed according to the following general procedure:

Into an autoclave made of stainless steel having an internal volume of 160 mL was initially charged a mixture of para-tolyl isocyanate (11 mL; 11.62 g; 0.087 mol) and propylene carbonate (47.40 mL; 57.05 g; 0.559 mol). Once the autoclave was sealed a low argon stream (20 L/min) was passed through the reactor and the reaction mixture heated to reaction temperature with stirring. Once a constant reaction temperature had been observed over a period of 5 minutes the in-situ IR measurement was initiated. The catalyst solution was subsequently injected into the reaction mixture in the reported amounts. The thus obtained reaction mixture was stirred at the relevant reaction temperature at a stirring speed of 500 rpm. If the intensity of the in-situ IR signal of the isocyanate group had fallen below the detection limits in a period of less than 40 minutes the reaction was terminated after a further 20 minutes by cooling the reactor to 25° C. and stopping the stirrer. Otherwise the reaction was terminated in the same way after one hour. The results are reported in Table 1.

Examples marked with an * are comparative examples.

TABLE 1

| | | | | | Activity: | Activity: | Selectivity | Selectivity | Selectivity | Selectivity |
|---|---|---|---|---|---|---|---|---|---|---|
| | | | | | Time | Time | for trimer | for trimer | for trimer | for trimer |
| | | | Degree | | until | until | formation | formation | formation | formation |
| | | | of | | conver- | conver- | at an iso- | at an iso- | at an iso- | at an iso- |
| | | Mol | depro- | | sion of | sion of | cyanate | cyanate | cyanate | cyanate |
| | | K | ton- | | 20% of | 50% of | conver- | conver- | conver- | conver- |
| | | per | ation | Catalyst | isocyan- | isocyan- | sion of | sion of | sion of | sion of |
| | Catalyst | mol | in | mol %/ | ate | ate | 20% in | 50% in | 90% in | 99% in |
| Example | precursor | cat. | % | wt % | in s | in s | % | % | % | % |
| 1-1* | acetate | 1 | 50 | 0.1/0.07 | 19.09 | 82.29 | 17.16 | 41.92 | 59.00 | 68.40 |
| 1-2* | 3- | 1 | 50 | 0.1/0.11 | 32.40 | 134.19 | 0.0 | 34.94 | 57.27 | 65.99 |
| | mercapto- | | | | | | | | | |
| | propionate | | | | | | | | | |
| 1-3 | 3- | 1.5 | 75 | 0.1/0.12 | 11.53 | 28.00 | 25.78 | 43.96 | 61.08 | 70.74 |
| | mercapto- | | | | | | | | | |
| | propionate | | | | | | | | | |
| 1-4 | 3- | 1.7 | 83.6 | 0.1/0.13 | 7.15 | 17.06 | 27.28 | 48.34 | 61.03 | 72.57 |
| | mercapto- | | | | | | | | | |
| | propionate | | | | | | | | | |
| 1-5 | 3- | 2 | 100 | 0.1/0.14 | 5.70 | 16.18 | 25.64 | 43.65 | 59.92 | 70.97 |
| | mercapto- | | | | | | | | | |
| | propionate | | | | | | | | | |
| 1-6 | 4-mercapto- | 2 | 100 | 0.1/0.15 | 4.73 | 15.09 | 7.40 | 31.55 | 53.23 | 62.16 |
| | butyrate | | | | | | | | | |
| 1-7 | thiosalicylate | 2 | 100 | 0.1/0.17 | 6.59 | 22.48 | 4.57 | 24.25 | 50.03 | 60.06 |
| 1-8* | S- | 1 | 100 | 0.2/0.3 | 28.09 | 139.46 | 0.0 | 27.78 | 62.29 | 70.07 |
| | methyl- | | | | | | | | | |
| | thiosalicylate | | | | | | | | | |
| 1-9* | 3-mercapto- | 1 | 50 | 0.1/0.15 | 58.21 | 279.30 | 0.0 | 13.14 | 50.05 | 59.82 |
| | propionate + | | | | | | | | | |
| | DBTL 9:1 | | | | | | | | | |
| | molar ratio | | | | | | | | | |
| 1-10* | 3-mercapto- | 2 | 100 | 0.1/0.17 | 9.44 | 33.32 | 17.36 | 25.22 | 50.06 | 65.28 |
| | propionate + | | | | | | | | | |
| | DBTL 9:1 | | | | | | | | | |
| | molar ratio | | | | | | | | | |

The degree of deprotonation is to be understood as meaning the percentage of Zerewittinoff-active protons removed from the acid upon which the catalyst molecule is based. Zerewittinoff-active protons are those that react with the Grignard reagent methylmagnesium iodide to form one molecule of methane per active proton.

Comparison of example 1-1 with examples 1-5 to 1-7 shows that the dipotassium salts of the mercaptoacids are superior to the potassium acetate (prior art) in terms of activity since the time for achieving a reported conversion is always lower.

Examples 1-2 and 1-8 compared to examples 1-5 to 1-7 show that it is advantageous when not only the carboxyl group but also the mercapto group is deprotonated.

A comparison of example 1-2 with example 1-9 and a comparison of example 1-5 with example 1-10 show that addition of DBTL (prior art) reduces activity, i.e. sole use of the deprotonated mercapto acid is advantageous over the prior art. The formation of trimers is desirable since they are advantageous for flame retardancy and heat resistance.

Example 1-5 shows that the dipotassium salt of 3-mercaptopropionic acid shows the greatest selectivity for trimer formation for all isocyanate conversions investigated. The comparisons of example 1-2 with example 1-9 and of example 1-5 with example 1-10 show that an addition of DBTL (dibutyltin dilaurate) has a disadvantageous effect on selectivity for trimer formation.

Examples 1-3 to 1-5 show that even at degrees of deprotonation of the catalyst in the range from ≧70% to ≦100% (examples 1-3 to 1-5) or from ≧80% to ≦100% (examples 1-4 to 1-5) a high selectivity for trimer formation is obtained for all isocyanate conversions investigated.

### Example Group 2

### Production of Polyurethane/Polyisocyanurate Foams

In the production of rigid foams the following compounds were employed:

| | |
|---|---|
| Polyesterpolyol | obtained from phthalic anhydride, adipic acid, |
| P1 | Monoethylene glycol and diethylene glycol, OH |
| | number 240 mg KOH/g |
| TCPP | tris(1-chloro-2-propyl)phosphate from Lanxess |
| | GmbH, Germany. |
| TEP | triethylphosphate from Lanxess GmbH, Germany. |
| Stabiliser B8443 | polyether-polysiloxane copolymer from Evonik. |
| Desmophen ® | polyetherpolyol based on trimethylolpropane and |
| V 657 | ethylene oxide having an OH number of 255 mg |
| | KOH/g according to DIN 53240 from Bayer |
| | MaterialScience AG, Leverkusen, Germany. |
| Additive 1132 | Polyesterpolyol from phthalic anhydride and |
| | diethylene glycol, OH-number 795 mg KOH/g |
| | from Bayer MaterialScience AG, Leverkusen, |
| | Germany. |
| Desmodur ® | polymeric polyisocyanate based on 4,4-diphenyl- |
| 44V70L | methane diisocyanate having an NCO content of |
| | about 31.5 wt % from Bayer MaterialScience AG, |
| | Leverkusen, Germany. |
| 3-MP | solution of 3-mercaptopropionic acid dipotassium |
| | salt (3-MP) (17.3 wt %) in DEG |

To produce the rigid foams the raw materials listed in table 2 except the polyisocyanate component were weighed into a paper cup, temperature-controlled to 23° C. and mixed using a Pendraulik laboratory mixer (e.g. Type LM-34 from Pendraulik) and volatilized blowing agent (n-pentane) was optionally supplemented. The polyisocyanate component (likewise temperature-controlled to 23° C.) was then added to the polyol mixture with stirring and the resultant reaction mixture was mixed for 8 s at 4200 rpm.

Examples marked with an * are comparative examples.

TABLE 2

| Example No. | 2-1* | 2-2 | 2-3* | 2-4 | 2-5* | 2-6 |
|---|---|---|---|---|---|---|
| Polyesterpolyol P1 (parts by weight) | 63.8 | 63.8 | 63.8 | 63.8 | 63.8 | 63.8 |
| TCPP (parts by weight) | 20.0 | 20.0 | 20.0 | 20.0 | 20.0 | 20.0 |
| TEP (parts by weight) | 5.0 | 5.0 | 5.0 | 5.0 | 5.0 | 5.0 |
| Desmophen ® V 657 (parts by weight) | 5.0 | 5.0 | 5.0 | 5.0 | 5.0 | 5.0 |
| Additive 1132 (parts by weight) | 2.2 | 2.2 | 2.2 | 2.2 | 2.2 | 2.2 |
| Stabiliser B8843 (parts by weight) | 4.0 | 4.0 | 4.0 | 4.0 | 4.0 | 4.0 |
| Potassium acetate, 10.0 wt % in DEG | 6.64 | — | 6.66 | | 7.18 | |
| (parts by weight) | | | | | | |
| 3-MP, 17.3 wt % in DEG (parts by | — | 7.20 | | 7.20 | | 7.80 |
| weight) | | | | | | |
| n-Pentane (parts by weight) | 17.1 | 17.5 | 17.1 | 17.5 | 18.1 | 18.5 |
| Desmodur ® 44V70L (parts by weight) | 196 | 201 | 196 | 201 | 214 | 221 |
| Index | 340 | 340 | 340 | 340 | 364 | 364 |
| Fibre time (s) | | | 105 | 40 | 100 | 39 |
| Core apparent density (kg/m³) | | | 37 | 39 | 37 | 40 |
| Mol % of catalyst based on employed | 0.5 | 0.5 | 0.5 | 0.5 | 0.5 | 0.5 |
| NCO groups | | | | | | |
| BVD test, | | | CI 5, | CI 5, | CI 5, | CI 5, |
| Flame height | | | 150 | 120-130 | 140-150 | 130 |
| | | | mm | mm | mm | mm |

Characterization of Reactivity of Catalyst 3-MP

FIG. 1 shows a plot of the rise height of the PUR/PIR foam versus time, measured for the foam recipe from example 2-2, where potassium acetate has been replaced by 3-MP as catalyst (K/S=2). In both cases the catalyst concentration was 0.5 mol% based on the employed isocyanate groups.

It was observed that the foam obtained using 3-MP achieved a greater rise height in a shorter time than the foam obtained using the comparative catalyst potassium acetate (Ac).

The inventive catalyst accordingly shows a higher activity. Furthermore, when using potassium acetate (example 2-1*) a reduced rate of increase in rise height (PIR kink) which is typically associated with onset of the trimerization reaction (isocyanurate formation) was observed after a time of >100 s.

Without wishing to be bound to a particular scientific theory the absence of the PIR kink when using 3-MP as catalyst is attributed to the trimerization reaction undergoing earlier onset and progressing simultaneously to the urethane formation in this case. The catalyst derived from 3-mercaptopropionic acid thus exhibits an increased relative reactivity for the formation of isocyanurate units compared to the comparative catalyst, potassium acetate.

FIG. 2 also confirms the increased activity of 3-MP for trimer formation through IR spectroscopic investigations. FIG. 2 shows a corresponding analysis of the reaction processes in the edge zone of the reacting foams in contact with a substrate at constant temperature (40, 70 and 120° C.). The experiments reflect the reaction behavior that the foam systems would show for example in contact with appropriately temperature-controlled covering layers in the production of metal panels.

Carbamate formation is activated virtually identically by both catalysts (FIG. 2).

FIG. 3 confirms temperature-dependent differences in the formation of trimer: at 40° C. and 70° C. 3-MP catalyses trimer formation markedly earlier and more strongly than Ac. At 120° C. a difference over time is still apparent but the final level achieved is comparable.

Characterization of Fire Behavior of Foams Produced with the Catalyst 3-MP

Identical catalyst concentrations of 0.5 mol% based on isocyanate groups employed and similar apparent densities were established for all foams. It is apparent that the foams activated with potassium acetate (examples 2-3* and 2-5*) have markedly longer fibre times, thus illustrating the lower activity compared to 3-MP (examples 2-4 and 2-6). Also, while all foams achieve the same combustibility of 5, the foams produced with the inventive catalyst 3-MP exhibit markedly lower flame heights.

1. A process for producing isocyanurates and isocyanurate-containing polyurethanes comprising reacting an isocyanate in the presence of a catalyst,
wherein:
the catalyst comprises the product of the reaction of a thiol group containing carboxylic acid with an alkali metal, alkaline earth metal, scandium-group or lanthanoid base,
wherein the reaction is performed in the absence of compounds comprising tin or lead,
wherein the degree of deprotonation of the catalyst is ≧50% to ≦100% and the H atoms present in carboxyl groups as well as the carboxylate groups and the H atoms present in thiol groups as well as the thiolate groups are considered when calculating the degree of deprotonation.

2. The process as claimed in claim 1, wherein the thiol group containing carboxylic acid comprises a thiol group and a carboxyl group.

3. The process as claimed in claim 2, wherein the thiol group containing carboxylic acid is selected from the group consisting of 2-mercaptoacetic acid, 3-mercaptopropionic acid, 4-mercaptobutyric acid, thiosalicylic acid, and combinations of any thereof.

4. The process as claimed in claim 1, wherein the degree of deprotonation of the catalyst is ≧70% to ≦100% and the H atoms present in carboxyl groups as well as the carboxylate groups and the H atoms present in thiol groups as well as the thiolate groups are considered when calculating the degree of deprotonation.

5. The process as claimed in claim 1, wherein the base for deprotonating the catalyst precursor is selected from the group consisting of an alkali metal, alkaline earth metal, scandium-group or lanthanoid hydride, an alkali metal, alkaline earth metal, scandium-group or lanthanoid alkoxide or an alkali metal, alkaline earth metal, and scandium-group or lanthanoid alkyl.

6. The process as claimed in claim 1, wherein the catalyst is present in the form of a solution or a suspension in a solvent before commencement of the reaction.

7. The process as claimed in claim 1, wherein the temperature at commencement of the reaction is ≧20° C. to ≦90° C.

8. The process as claimed in claim 1, wherein the isocyanate is a polyisocyanate.

9. The process as claimed in claim 8, wherein the reaction is further performed in the presence of a polyol.

10. The process as claimed in claim 9, wherein the reaction is further performed in the presence of a physical blowing agent and/or a chemical blowing agent.

11. The process as claimed in claim 10, wherein the reaction is performed at an NCO index of ≧200.

12. A polyurethane/polyisocyanurate foam produced by a process as claimed in claim 10.

13. The polyurethane/polyisocyanurate foam as claimed in claim 12, wherein the polyurethane/polyisocyanurate foam further has a combustibility index CI of 5 and a flame height of ≦135 mm, in each case determined in the BVD test.

14. A thermal insulation element comprising a polyurethane/polyisocyanurate foam as claimed in claim 12.

15. The process as claimed in claim 3, wherein the degree of deprotonation of the catalyst is ≧70% to ≦100% and the H atoms present in carboxyl groups as well as the carboxylate groups and the H atoms present in thiol groups as well as the thiolate groups are considered when calculating the degree of deprotonation.

16. The process as claimed in claim 15, wherein the base for deprotonating the catalyst precursor is selected from the group consisting of an alkali metal, alkaline earth metal, scandium-group or lanthanoid hydride, an alkali metal, alkaline earth metal, scandium-group or lanthanoid alkoxide or an alkali metal, alkaline earth metal, and scandium-group or lanthanoid alkyl.

17. The process as claimed in claim 16, wherein the catalyst is present in the form of a solution or a suspension in a solvent before commencement of the reaction.

18. The process as claimed in claim 17, wherein the temperature at commencement of the reaction is ≧20° C. to ≦90° C.

19. The process as claimed in claim 18, wherein the isocyanate is a polyisocyanate.

20. A thermal insulation element comprising a polyurethane/polyisocyanurate foam as claimed in claim 13.
